(19) United States
(12) Patent Application Publication
(10) Pub. No.: US 2013/0066269 A1
(43) Pub. Date: Mar. 14, 2013

(54) RETRACTABLE SAFETY SYRINGE

(71) Applicants: Hsueh-Jung LU, Taoyuan County (TW); Chin-Shu Tsai, Taoyuan County (TW)

(72) Inventors: Hsueh-Jung LU, Taoyuan County (TW); Chin-Shu Tsai, Taoyuan County (TW)

(21) Appl. No.: 13/228,860

(22) Filed: Sep. 9, 2011

(51) Int. Cl. A61M 5/50

(52) U.S. Cl. 604/110

(57) ABSTRACT

A retractable safety syringe comprises a needle body, a syringe barrel, and a pusher plunger. The needle body has a needle and a needle seat having an upper needle seat and a lower needle seat; the upper needle seat is used for fixing the needle thereon and is provided with a connecting portion on the bottom; and the removable lower needle seat can be embedded to the upper needle seat from below. The pusher plunger has a piston in the front end thereof; the piston has a connecting member, which can connect to the connecting portion on the bottom of the upper needle seat, so that after injection, by applying a force to press and deform the piston till connecting to the connecting portion, the needle body can depart from the needle socket and the abutment and can be drawn back to the interior of the syringe barrel.

### BACKGROUND OF THE INVENTION

1. Field of the invention

The present invention relates to a retractable safety syringe, particularly to an engaging structure of a syringe barrel and a retractable needle body, which is able to sustain the syringe needle effectively and stably. The syringe needle can be drawn into the interior of the syringe barrel after injection, and therefore is safer for disposal or process. The engaging structure is suitable to be used for medical syringes or similar article.

2. Background of the Invention

Currently, the syringes for subcutaneous injections have been widely used in medicine. They are usually used for once and are disposed right after use. It is convenient and hygienic, and can prevent the infection via the syringe needle. However, the exposed needle of the disposed syringes may prick medical staffs by accidents, and leads to injury or infection. Other people can be pricked by the sharp needle as well, if the disposed syringes are not properly handled and are disposed carelessly. People who take care of the medical waste may be injured by the disposed syringe needle as well. Therefore retractable safety syringes are produced. The syringe needle can be drawn back into the interior of the syringe barrel after use and thus can be hidden inside the syringe barrel. The volume of the syringe can be reduces in the mean time.

However, the connecting structure of the syringe needle and the syringe barrel of a retractable safety syringe is very important. Because of the difference of the manufacturing tolerance, sometimes the syringe needle can not be inserted in and combined to the syringe barrel easily, and sometimes the connection of the syringe needle to the front of the syringe barrel is too loose and the syringe needle can not be fastened merely by the flange provided on the needle seat of the needle body to wedge to the inner side of the front end of the syringe barrel. Besides, without a proper mechanism to prevent the improper connection of the pusher plunger and the syringe needle during transportation, the syringe may become ineffective.

In view of these, the inventor devoted to develop a better structure which can improve the connection of the syringe needle and the syringe barrel.

### SUMMARY OF THE INVENTION

The primary object of the present invention is to provide a retractable safe syringe, which has simple structure, is able to sustain the needle body effectively and stably, and needle body thereof can be drawn into the interior of the syringe barrel after injection.

To achieve the above objects, the retractable safety syringe of the present invention is comprised of a needle body, a syringe barrel, and a pusher plunger. Wherein, the needle body comprises a needle and a needle seat. The needle seat comprises an upper needle seat and a lower needle seat. The upper needle seat is for fixing the needle thereon, and the removable lower needle seat is fixed to the upper needle seat from below. On the bottom of the upper needle seat is provided with a connecting portion. The syringe barrel is a hollow cylinder having in the front end thereof a needle socket for embedding the needle body, so that the needle seat of the needle body can be held in the needle socket. In the inner side of the needle socket is provided with an abutment for the needle seat of the needle body to abut against it. The pusher plunger can be pushed into the syringe barrel from below. In front of said pusher plunger is provided with a piston. The piston is connected to a connecting member, which can be attached to the connecting portion on the bottom of the upper needle seat, so that after injection, by applying a force to press and deform the piston till the connecting member attaching to the connecting portion, the needle body can depart from the needle socket and the abutment and be drawn back to the interior of the syringe barrel.

In an embodiment, the pusher plunger has a diameter-shrinking portion. When the diameter-shrinking portion of the pusher plunger is drawn back near the end of the syringe barrel, the pusher plunger can be snapped off by applying a force. The upper needle seat and the lower needle seat should be provided with corresponding wedge and socket. The lower needle seat is provided with a holding space for holding the connecting portion of the upper needle seat.

To enable a best understanding of the present invention, the disclosure and the features of the present invention will be described in the following detailed description of the preferred embodiments in reference to the accompanying drawings.

### BRIEF DESCRIPTION OF DRAWINGS

FIG. 1 is a schematic view of the needle seat of an embodiment of the present invention in a state before assembly.

FIG. 2 is a schematic view of the needle seat of an embodiment of the present invention in a state after assembly.

FIG. 3 is a schematic view of the pusher plunger of an embodiment of the present invention in a state before entering the syringe barrel.

FIG. 4 is a schematic view showing a state after connecting the connecting member to the connecting portion of the lower needle seat of an embodiment of the present invention.

FIG. 5 is a schematic view showing an embodiment of the present invention in a state after drawing back the pusher plunger.

### DETAILED DESCRIPTION OF THE PREFERRED EMBODIMENT

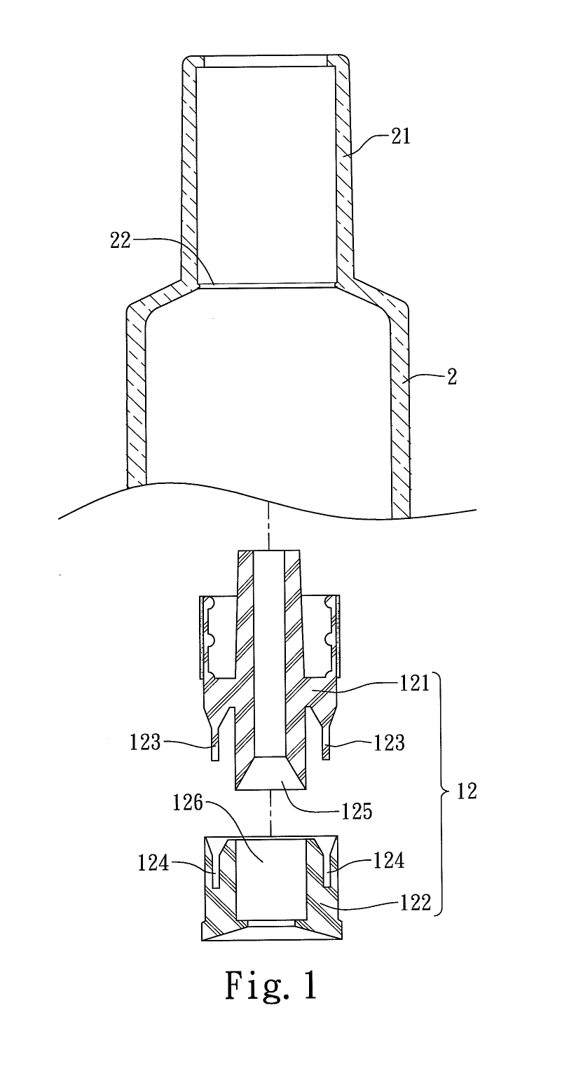

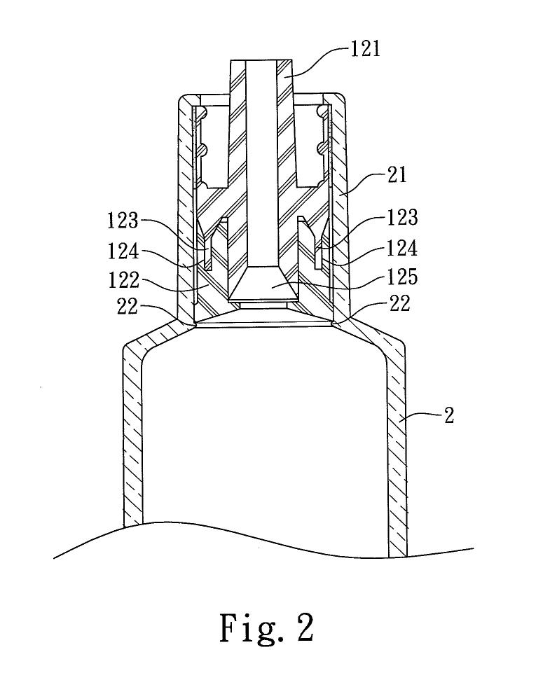

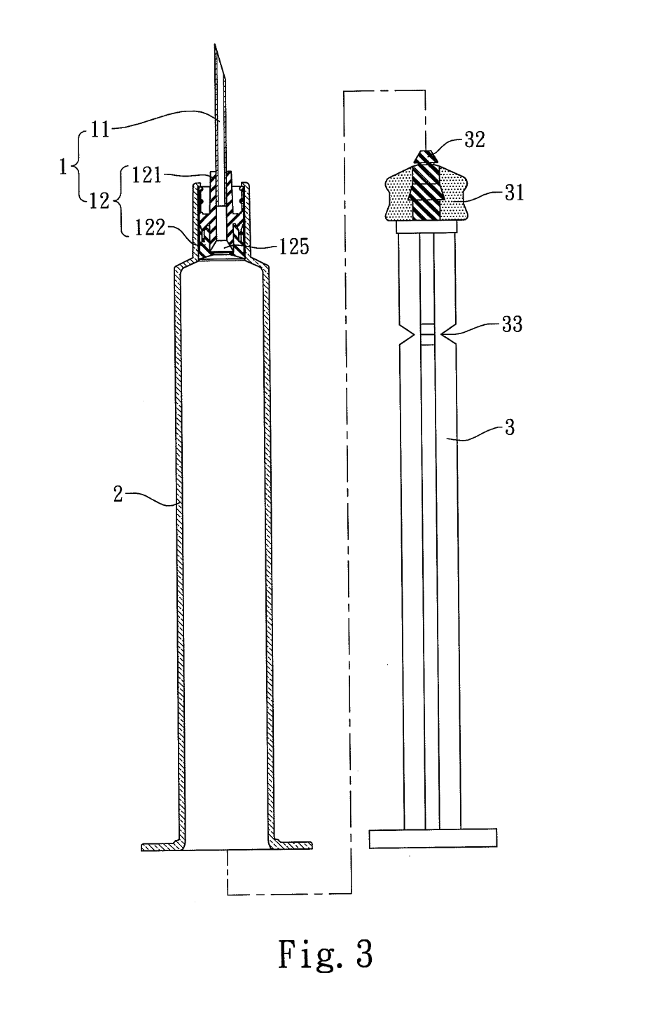

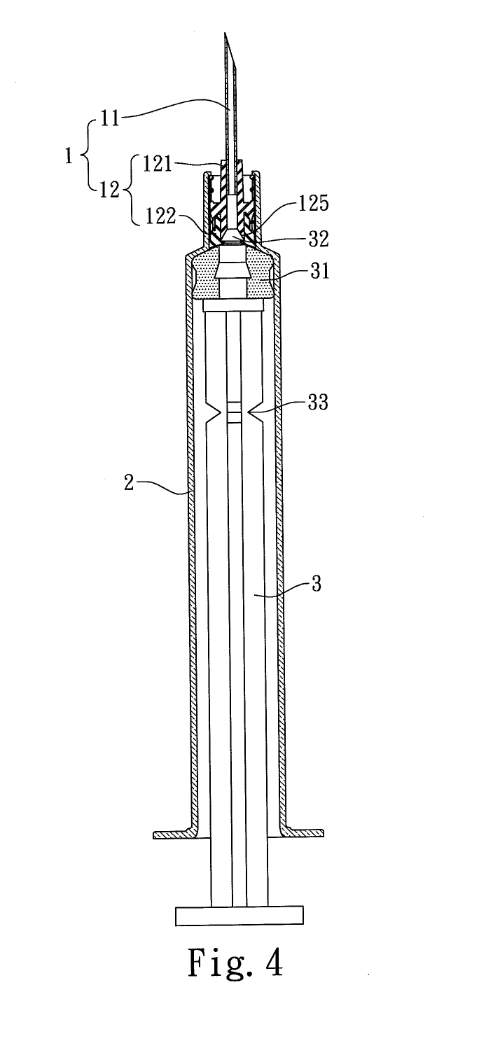

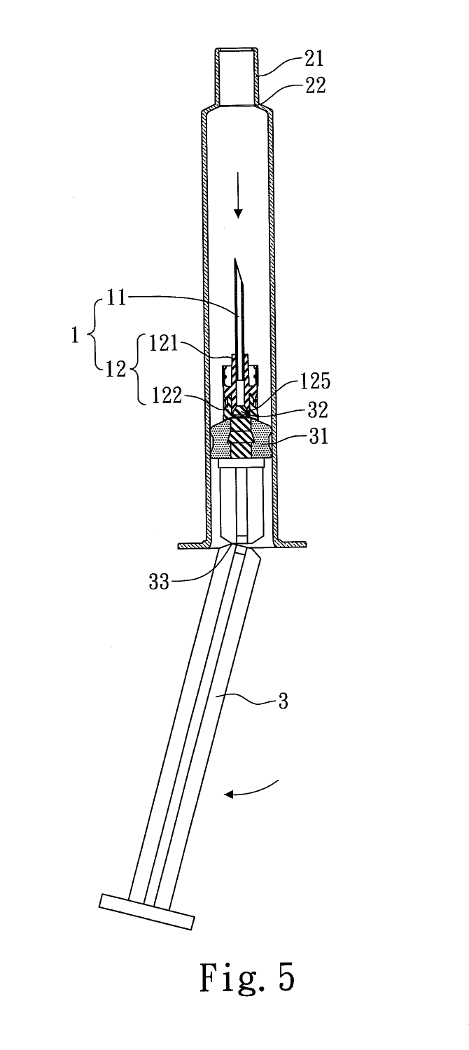

Referring to FIGS. 1-5, which depict an embodiment of a retractable safety syringe of the present invention, the syringe comprises a needle body 1, a syringe barrel 2, and a pusher plunger 3.

The needle body 1 comprises a needle 11 and a needle seat 12; the needle seat 12 comprises an upper needle seat 121 and a lower needle seat 122; the needle 11 can be fixed to the upper needle seat 121 from below; the upper needle seat 121 has a wedge 123, which can be fastened to the corresponding socket 124 in the lower needle seat 122, and therefore the removable lower needle seat 122 can be fixed to the upper needle seat 121; on the bottom of the upper needle seat 121 is provided with a connecting portion 125; in the lower needle seat 122 is provided with a holding space 126 for holding the connecting portion 125 of the upper needle seat 121.

The syringe barrel 2 is a hollow cylinder having in the front end thereof a needle socket 21 for embedding the needle body 1, so that the needle seat 12 of the needle body 1 can be held in the needle socket 21; in the inner side of the needle socket 21 is provided with an abutment 22 for the needle seat 12 of the needle body 1 to abut against it.

The pusher plunger 3 can be pushed into the syringe barrel 2 from below; in front of the pusher plunger 3 is provided with a piston 31; the piston 31 is connected to a connecting member 32, which can be attached to the connecting portion 125 on the bottom of the upper needle seat 121; the connecting member 32 is an extruded tenon; the pusher plunger 3 has a diameter-shrinking portion 33, so that the pusher plunger 3 can be snapped off by applying a force.

In an embodiment, after the upper needle seat 121 and the lower needle seat 122 entering the syringe barrel 2 from below subsequently, and after passing through the abutment 22 on the bottom of the needle socket 21, through the wedge 123 of the upper needle seat 121 and its corresponding socket 124 of the lower needle seat 122, the removable lower needle seat 122 can be fixed to the upper needle seat 121 from below and assemble the needle seat 12. The needle seat 12 of the needle body 1 can be firmly embedded in the needle socket 21 through the abutment 22 in the inner side of the needle socket 21 and therefore a sealed state is created during the process of drawing in medicine and injection.

After the injection process, a force can be applied to press and deform the piston 31 till the connecting member 32 protruding the lower needle seat 122 and attaching to the connecting portion 125 of the upper needle seat 121. After drawing back the pusher plunger 3, the connecting member 32 of the piston 31 in front of the pusher plunger 3 can jointly draw the lower needle seat 122 together with the upper needle seat 121 apart from the support of the abutment 22, which makes the needle body 1 departing from the needle socket 21 and the abutment 22 and being drawn back to the interior of the syringe barrel 2. When the diameter-shrinking portion 33 of the pusher plunger 3 is drawn back near the end of the syringe barrel 2, the pusher plunger 3 can be snapped off by applying a force, and it can be recycled and reused, which effectively reduce waste and cost.

The above description shows embodiments of the present invention. Many modifications and variations may be made by those skilled in the art from the teachings disclosed hereinabove. Therefore, it should be understood that any modification and variation equivalent to the spirit of the present invention be regarded to fall into the scope defined by the appended claims.

1. A retractable safety syringe, comprising:
a needle body, including a needle and needle seat, wherein said needle seat includes an upper needle seat and a lower needle seat, said upper needle seat is for fixing said needle thereon, said lower needle seat is fixed to the upper needle seat from below, on the bottom of the upper needle seat is provided with a connecting portion;
a syringe barrel, being a hollow cylinder having in the front end thereof a needle socket for embedding the needle body, so that the needle seat of the needle body can be held in said needle socket, in the inner side of said needle socket locates an abutment for the needle seat of the needle body to abut against it; and
a pusher plunger, being pushable into the syringe barrel from below, in front of said pusher plunger is provided with a piston, said piston is disposed with a connecting member, wherein the connecting member is an extruded tenon on a front end of the piston for passing through the lower needle seat and connecting to the connecting portion on the bottom of the upper needle seat by applying a force to press and deform the piston after injection so that the needle body can depart from the needle socket and the abutment and be drawn back to the interior of the syringe barrel by drawing back the pusher plunger.

2. The retractable safety syringe as claimed in claim 1, wherein said pusher plunger has a diameter-shrinking portion, when said diameter-shrinking portion of said pusher plunger is drawn back near the end of the syringe barrel, said pusher plunger can be snapped off by applying a force.

3. The retractable safety syringe as claimed in claim 1, wherein said upper needle seat and said lower needle seat are provided with corresponding wedge and socket.

4. The retractable safety syringe as claimed in claim 1, wherein said lower needle seat is provided with a holding space for holding the connecting portion of the upper needle seat.

5. The retractable safety syringe as claimed in claim 3, wherein the upper needle seat is disposed with a downwardly projecting wedge on a bottom thereof, and the lower needle seat is disposed with a socket on a top thereof for fastening the wedge.
